SEQUENCE LISTING (2 sequences)

SEQ ID NO: 1 - protein, 34 aa, Virus
DAATATRGRSAASRPTERPRAPARSASRPRRPVE

SEQ ID NO: 2 - protein, 11 aa, Artificial
  The sequence is an example of peptide sequences (R)n, wherein R=Arginine and n is no less than 4, as stated in Claim 3. In the example, n=11.
RRRRRRRRRRR

(19) United States
(12) Patent Application Publication
(10) Pub. No.: US 2015/0023932 A1
(43) Pub. Date: Jan. 22, 2015

(54) ENGINEERED PEPTIDE (EP)-DIRECTED PROTEIN INTERCELLULAR DELIVERY SYSTEM AND USES THEREOF

(71) Applicants: KE-KE FAN, Fremont, CA (US); Jing Bian, Fremont, CA (US)

(72) Inventors: KE-KE FAN, Fremont, CA (US); Jing Bian, Fremont, CA (US)

(21) Appl. No.: 13/943,793

(22) Filed: Jul. 17, 2013

(51) Int. Cl. C07K 14/48; A61K 38/18; C12N 5/074; C07K 14/47; C12N 5/0797

(52) U.S. Cl. CPC C07K 14/48; C07K 14/47; C12N 5/0623; C12N 5/0696; A61K 38/185; C07K 2319/036; C07K 2319/10; C12N 2506/45; C12N 2506/02; C12N 2501/603; C12N 2501/602; C12N 2501/604; C12N 2501/606
USPC 424/93.21; 530/350; 435/69.7; 435/377; 435/366; 435/357; 435/353; 435/183

(57) ABSTRACT

The present invention provides an intercellular protein delivery system comprising an engineered peptide (EP), composed of secretion part (SP) and nuclear translocation part (NTP), a functional or therapeutic protein (FP), cells that express the fusion proteins and cells that accept the fusion proteins. The system can be used in vivo or in vitro to sustainably supply proteins of interest for cellular reprogramming, cellular differentiation and cell-based protein therapies.

### CROSS-REFERENCE TO RELATED APPLICATION

This application claims priority under 35 U.S.C. sctn. 119 to U.S. Provisional Application No. 61/672,400, filed Jul. 17, 2012, the contents of which are incorporated herein by reference.

### FIELD OF THE INVENTION

The present invention concerns a protein intercellular delivery system and their applications. In particular embodiments, the invention relates to fusion proteins comprising engineered peptides for both secretion from the cells and translocation into the cells thereof together with other functional or therapeutic proteins; and to methods for their preparations and uses. In particular embodiments, the invention relates to fusion proteins with engineered peptides for cell reprogramming into pluripotency and for pluripotent stem cells differentiating into specific functional cells such as cardiomyocytes and neural cells. In particular examples the invention for cell reprogramming into pluripotency relates to fusion proteins having both pluripotent transcription factors such as Oct4, Sox2, Klf4 or c-Myc functionality, and engineered peptides as described in the claims for intercellular delivery. In particular examples of pluripotent stem cell differentiating into cardiac cells and neural cells, the invention relates to fusion protein having cardiac key transcription factors such as Tbx5, MEF2c, GATA4 or Isl1, and engineered peptide; or neural key transcription factors such as NeuroD1, Pax6 and LHX2, and engineered peptide, as described in the claims. In another particular embodiment, the invention relates to the uses of the sustained delivery of therapeutic proteins in cell-based therapies of intracellular proteins or antibodies. Other aspects of the invention will be apparent from the description and claims.

### BACKGROUND OF THE INVENTION

Generally cell penetrating peptides (CPP) members consist of positive-charged short peptide sequences that have the ability to cross the plasma membrane into the cell interior in a seemingly energy and receptor-independent manner. Comparing to conventional virus gene delivery system CPP protein delivery technology with the advantage of low toxicity has great clinical application potentials. However, most CPP members with high efficiency in cell translocation such as TAT or 7-9 repeated arginine polypeptides widely used in cancer research or other scientific areas has very poor capability to secrete from the expressing cells.

This invention is in design and development of an engineered peptide containing both secretion part and translocation part that could facilitate transporting from expressing cells into recipient cells which include a process of proteins leaving expressing cells and a process of proteins entering recipient cells.

Furthermore, the present invention concerns about its important applications in cell reprogramming and cell differentiation for regenerative medicine. Recent breakthrough technology of reprogramming somatic cells into induced pluripotent stem (iPS) cells, by enforced expression of few key transcription factors, provides an autologous source of pluripotent stem cells to regenerate healthy and functional end-organ cells with great clinical prospects (Takahashi and Yamanaka, 2006; Yu et al., 2007). However, the genes involved and the viral integration into host genome increases the risk of tumorogenecity. For clinical translation of this exciting technology to be achieved it is crucial that iPS cells be generated using non-integrating methods (Kim et al, 2009).

In the particular examples of the invention, making chimeric proteins that include the reprogramming factor protein(s) may be Sox2, Klf4, Oct3/4, c-Myc linked recombinantly or chemically to the engineered peptide helps facilitate the introduction of these proteins into the target cells for direct protein reprogramming. Accordingly, the present method of inducing pluripotent stem cell (iPS) formation avoids the use of viral or DNA-based expression vectors or the expression of reprogramming factor genes within target cells, which are known to be harmful to the host target cell and cause cancer.

Over a decade ago, Wilmut et al showed that adult somatic cells could be reprogrammed back to an undifferentiated embryonic state using somatic cell nuclear transfer (SCNT) (Wilmut et al., 1997). However, since that time attempts to generate patient-specific cells using SCNT have proven unsuccessful (Chung et al., 2009; French et al., 2008).

In 2006, Yamanaka et al established a method of using four transcription factors, Oct4, Klf4, Sox2, and c-Myc, to reprogram murine somatic cells to induced pluripotent stem cells (iPSCs) (Takahashi and Yamanaka, 2006). Yu et al generated human iPSCs subsequently by using similar genetic manipulation (Takahashi et al., 2007; Yu et al., 2007). Although the therapeutic potential of iPS cells has been demonstrated in animal models of sickle cell anemia and Parkinson's disease (Hanna et al., 2007; Wernig et al., 2008), these cells contain multiple viral vector integrations that make them unsuitable for human clinical trials.

Current differentiation approach from pluripotent stem cells includes direct differentiation without forming embryonic body (EB) or EB derived differentiation. Both of those methods need to orchestrate adding different small molecules of chemicals and inhibitors following differentiation stages to induce efficient differentiation. However, almost all the small molecules for differentiation are very expensive and the differentiation process is time-consuming (Minami I, Cell Reports, 2012; Chambers S M, Nature Biotechnology, 2009).

The uses of cell penetrating peptides to deliver therapeutic proteins to treat various pathological conditions, such as cancer, inflammatory diseases, oxidative stress-related disorders, diabetes and brain injury, have been investigated by other investigators (Shiraishi T, Chem. Bio., 2005; Tan M L, Peptides, 2009); however, the delivery efficiency and the therapeutic efficacy of proteins were not significantly improved as expected, most likely due to they are cell “penetrating” peptides, not cell “secreting” peptides.

The current invention provides a sustainable and nontoxic source of proteins of interest instead of purified proteins which otherwise often exert cellular toxicity at a high concentration against target cells. The current invention of engineered peptides comprising secretion part and translocation part has another advantage over current cell penetrating peptides that is facilitating the secretion of expressed proteins from cells. That is, our engineered peptide works as both cell “penetrating” peptide and cell “secreting” peptide. The current invention enables a high efficacy of transporting functional factors or therapeutic proteins between cells and has a great potential for clinical use.

### DESCRIPTION OF TH RELATED ARTS

In 2009, in order to avoid the use of genetic materials and thus the potential for unexpected genetic modifications in the target cells, Ding et al reported the generation of protein-induced pluripotent stem cells (piPSCs) from murine embryonic fibroblasts using recombinant cell-penetrating reprogramming proteins; subsequently Kim et al reported the generation of stable iPS cells from human fibroblasts by directly delivering four reprogramming proteins (Oct4, Sox2, Klf4, and c-Myc) fused with a cell penetrating peptide (CPP).

One main reason for the low efficiency is that the reprogramming factors were not provided continuously and thus were in short supply. Second, delivering the reprogramming proteins directly into cells has to involve the processes of solubilization, refolding and purification of the recombinant proteins expressed in E. coli. In addition, the induction medium containing the recombinant proteins also has to be replaced several times due to the toxicity. Even with repeated protein treatment cycles which could improve the short supply of the reprogramming factors, the efficiency of iPS generation was still significantly lower (about 0.001% of input cells, Kim et al, 2009), compared to virus-based protocols (about 0.01% of input cells) (Park et al., 2008; Takahashi et al., 2007; Yu et al., 2007).

Herein we use a system that sustainably delivers the reprogramming factors in situ released directly from source cells thus waive the processes of solubilization, refolding and purification of the recombinant proteins. Also, we use the specifically engineered peptide sequence to significantly facilitate the transportation of the reprogramming factors in and out of target cells. Thus our system significantly increases the delivery efficiency to a level of being able to producing clinical-grade induced pluripotent stem cells in a large-scale. Our system not only eliminates the potential risks associated with the use of viruses, DNA transfection, and potentially harmful chemicals, and thus provides a safe and efficient source of normal or patient-specific induced pluripotent stem cells for regenerative medicine. In addition, the present system provides an efficient protein delivery into a broad variety of cell types such as skin fibroblasts, cardiac fibroblasts, adult mesenchymal cells, IMR90 cells for cell reprogramming; stem cells such as embryonic stem cells and induced pluripotent stem cells for cell differentiation; and cancer cells such as DU145 (prostate), C32 (melanoma) and A541 (lung) for cell-based protein or antibody therapies.

### SUMMARY OF THE INVENTION

In the present invention of the sustained protein intercellular delivery system, wherein an engineered peptide is developed comprising both cell secretion sequences and cell translocation sequences. In some embodiment, the nuclear translocation part of the engineered peptide is screened, identified and composed from the groups consisting of nuclear transduction domain and protein secretion part. Nuclear transduction domain includes, but not limited to, Drosophila homeoprotein antennapedia transcription protein (AntHD) (Derossi D, The Journal of Biological Chemistry, 1994), the HIV-1 transcriptional activator TAT protein (Green M, 1988, Cell; Lo SL, Biomaterials, 2008), Kaposi FGF signal sequence (kFGF), protein transduction domain-4 (PTD4), Penetratin, M918, Transportan-10, a nuclear localization sequence, a PEP-1 peptide; an amphipathic peptide; a delivery enhancing transporter, peptide sequence comprising at least 4 or more continuous arginines (Gautam A, CPPsite, Database (Oxford), 2012), and Xentry, a newly class of cell penetrating peptide (Montrose K, et al., Scientific Reports, 2013). The secretion part of the engineered peptide (Sequence ID No. 1) is designed based on Elliott's study (Elliott G, O'Hare P; Cell, 1997)

In some embodiment, non-secreted proteins such as transcription factors recombinantly connected with engineered peptide could make intercellular delivery.

In some embodiment, sustained protein delivery system is developed, which is consisted of transfected cells expressing fusion proteins, cell insert where transfected cells are plated, and fusion proteins comprising protein of interest and an engineered peptide that facilitates secretion from expressing cells and translocation into somatic cells being attached on the bottom well.

According to one aspect of the present invention, the transfected cell is expressing a fusion protein of a non-secreted protein such as a non-secreted transcription factor and an engineered peptide.

In some embodiment, the method comprising: contacting the non-pluripotent cell on the bottom well with one or more transfected cells expressing a transcription factor fused to an engineered peptide culturing in the cell insert; wherein the transcription factor is selected from the group consisting of a Klf polypeptide, an Oct polypeptide, a Myc polypeptide, and a Sox polypeptide.

In some embodiments, the engineered peptide is a peptide sequence comprising secretion part (for example, SEQ ID NO.1) and nuclear localization part (for example, SEQ ID NO. 2).

The present invention related to a safe, effective, and non-integrating strategy for reprogramming cell fate into pluripotency based on administration of sustained cell based delivery of functional proteins with an engineered peptide. Compared to other protein based reprogramming approach the present invention utilized a unique cell-based and sustainable protein delivery system to reprogram somatic cells into pluripotency so as to omit the additional step for preparation and isolation of recombinant proteins. We show that this approach can provide sustained protein delivery over several weeks and reprogram mouse, rat and human somatic cells to pluripotency with efficiencies that greatly surpass established non-integration protocols. This technology represents a safe, efficient strategy for somatic cell reprogramming and directing cell fate that has broad applicability for basic research, disease modeling, drug discovery and regenerative medicine.

In order to induce the epigenetic reprogramming of the cell into a pluripotent stem cell, the reprogramming factor protein(s) may be Sox2, Klf4, Oct3/4, c-Myc, or any proteins with reprogramming activity. Accordingly, the present method of inducing pluripotent stem cell (iPS) formation avoids the use of viral or DNA-based expression vectors or the expression of reprogramming factor genes within target cells, which are known to be harmful to the host target cell and often cause cancers.

Yet another aspect of the invention relates to a method that comprises at least one protein delivery vehicle expressing a transcription factor-EP (engineered peptide) fusion protein. A variety of direct reprogramming or direct differentiation kit may be introduced by the invention to a particular cell type produced with sustained protein intercellular delivery of different transcription factors with the engineered factor. Examples of cell type produced from this sustained protein delivery system of the present invention include, but are not limited to, motor neurons or other differentiated neural cells, cardiomyocytes, and insulin-producing beta-cells.

Another aspect of the invention relates to a method of using the engineered peptides to sustainably deliver therapeutic proteins or antibodies in cell-based protein or antibody therapies. The invention opens an avenue for cell-based therapeutic proteins to be secreted from donor cells, and enter target cells and bind to the intracellular targets for therapeutic purpose.

Another aspect of the invention relates to a method of using EP to sustainably deliver the transcription factors to reprogram mouse, rat and human somatic cells into induced pluripotent stem cells. And somatic cells include human mesenchymal stem cells, human IMR90, rat mesenchymal stem cells, rat skin fibroblasts, rat cardiac fibroblasts, and mouse mesenchymal stem cells.

The present invention relates to methods of using engineered peptides (EPs) to direct non-secreted protein intercellular delivery and the uses of the methods in cellular reprogramming, cellular differentiation and cell-based protein or antibody therapies. EPs contain two functional fragments: one for secretion of a protein from cells and the other for delivery of the protein into cells and thus facilitating non-secreted protein transport between cells. The current invention provides the first kind of engineered peptides with both secretion fragment and nuclear translocation fragment. Uses of EPs include cell-based sustained protein delivery of non-secreted transcription factors into somatic cells for reprogramming into pluripotent stem cells and for direct differentiation from pluripotent stem cells into other cell types. Uses of EPs also include the sustained delivery of therapeutic proteins or antibodies through in vivo transplanted cells.

The current invention waives the process of purifying and preparing proteins and avoids the cellular toxicity arouse by the use of a high concentration of purified proteins. The invention significantly improves the process and efficacy of delivering proteins between cells and enables the clinical-scale productions of induced pluripotent stem cells and differentiated cells. The invention also provides a great potential for improved clinical therapeutic efficacy of cell-based protein therapy.

### BRIEF DESCRIPTION OF THE DRAWINGS

FIG. 1. (A) Plasmid construction of engineered peptide (EP) containing protein secretion part (SP) and nuclear translocation part (NTP) (B) Diagram of transportation of EP fused with functional proteins between the cells. Engineered peptide fused with functional proteins secrets from an expressing donor cell (a) into a surrounding recipient cell (b) and localize into the nucleus.

FIG. 2. Sustained delivery of transcription factors with EP to induce mouse somatic cells reprogramming into induced pluripotent stem (iPS) cells. (A) Western blot analysis of fusion proteins EP fused with 4 key transcription factors Oct4, Sox2, Klf4 and C-myc expressed in 293 cells. (B) Composition of sustained protein intercellular delivery system for cellular reprogramming and cell differentiation. 293 cells transfected with fusion proteins of pluripotent transcription factors and EP are plated on the cell insert, and contacting cells are plated on the bottom well. (C) Quantification of each transcription factor (Oct4, Sox2, Klf4 or Cmyc) expressed in each cell population in the sustained protein delivery systems using flow cytometry analysis of intercellular delivery of Oct4, Sox2, Klf4 and Cmyc fused with EP from transfected 293 cells in the cell insert into mouse mesenchymal stem cells (mMSCs) on the bottom well.

FIG. 3. Mouse protein-based induced pluripotent stem (piPS) cell colonies were initially observed around day 30-35. (A) Representative phase contrast image of mouse mesenchymal stem cells and piPS at P0, P1 and P2 are shown. piPS cells sustain long term and homogenous self-renewal under conventional mESC growth condition. (B) The long-term expanded piPS cells grow as compact and domed colonies that express strong AP, a typical pluripotency marker including Oct4, Sox2, Nanog and SSEA-1. DAPI staining was performed to visualize the nuclei, and the images were merged. (C) Reprogramming efficiency of mouse piPS with sustained protein delivery system.

FIG. 4. (A) The pluripotency of mouse piPS cells. piPS cells can effectively differentiate in vitro into cells in the three germ layers, including characteristic neurons (TUJ1+), mature cardiomyocytes (Sacrometric actinin), definitive endoderm cells (Sox17). Images were merged with DAPI staining (B) PCR analysis of EP plasmid non-integrated into piPS cells. Oct4, Sox2, Klf4 and Cmyc did not integrate into the Chromosomes of reprogrammed cells and iPS cells

FIG. 5. Rat iPS cells generated with EP. (A) Rat protein based induced pluripotent stem cell colonies were initially observed in 6 days after 9 days EP based transduction. AP live fluorescence staining showed AP positive clone in rat iPS cells. (B) Reprogramming efficiency of rat iPS cells with EP delivery system.

FIG. 6. Human iPS generation with protein based direct reprogramming with engineered peptides: a) IMR-90 cells are reprogrammed into human piPS cells. b) Human piPS cells are characterized with AP staining (c) Human piPS cells reprogramming efficiency.

FIG. 7. Engineered peptide based neural differentiation. Mouse iPS cells were plated on the bottom well, and one million of 293 cells transiently transfected with EP:PAX6 were plated on the top well insert. After coculturing for 3 days, miPS cells on the bottom well were observed with change of morphology in neural rossette (A) and further differentiated into neurosphere (B) and further differentiated into neural epithelial progenitor stem cells with NE positive markers (C).

FIG. 8. Engineered peptide based cardiac differentiation: Rat MSCs were plated on the bottom well, and one million of 293 cells transiently transfected with EP:GATA4 were plated on the top well insert. After coculturing for 7 days, rat MSCs on the bottom well were observed with change of morphology (A) and analyzed with flow cytometry for its GATA4 expression (B) and its genomic means of GATA4 expression (C).

FIG. 9. Myogenic differentiation. One million of rat cardiac fibroblasts were plated on the bottom wells, and one million of 293 cells transiently transfected with EP:GATA4 were plated on the top well insert. After coculturing for 7 days, rat cardiac fibroblasts on the bottom well were observed with change of morphology similar like skeletal myoblasts and analyzed with immunocytochemistry for its muscle actin expression.

FIG. 10. Transplantation of mesenchymal cells expressing Neurotrophin 3 (NT3)-EP fusion proteins in a spinal cord injury (SCI) model. Compared to the delivery of NT3 alone, the delivery of NT3 fused with EP showed significantly improved therapeutic effect in vivo.

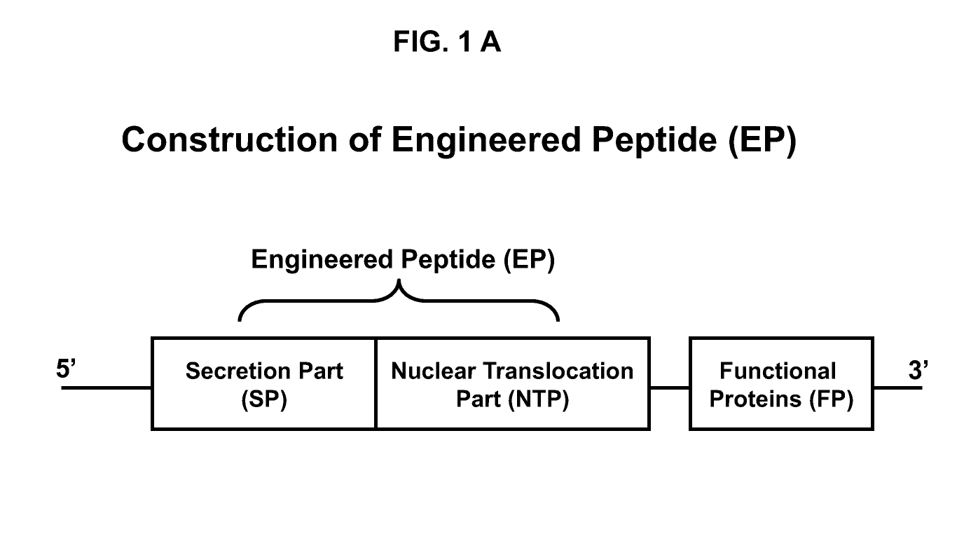

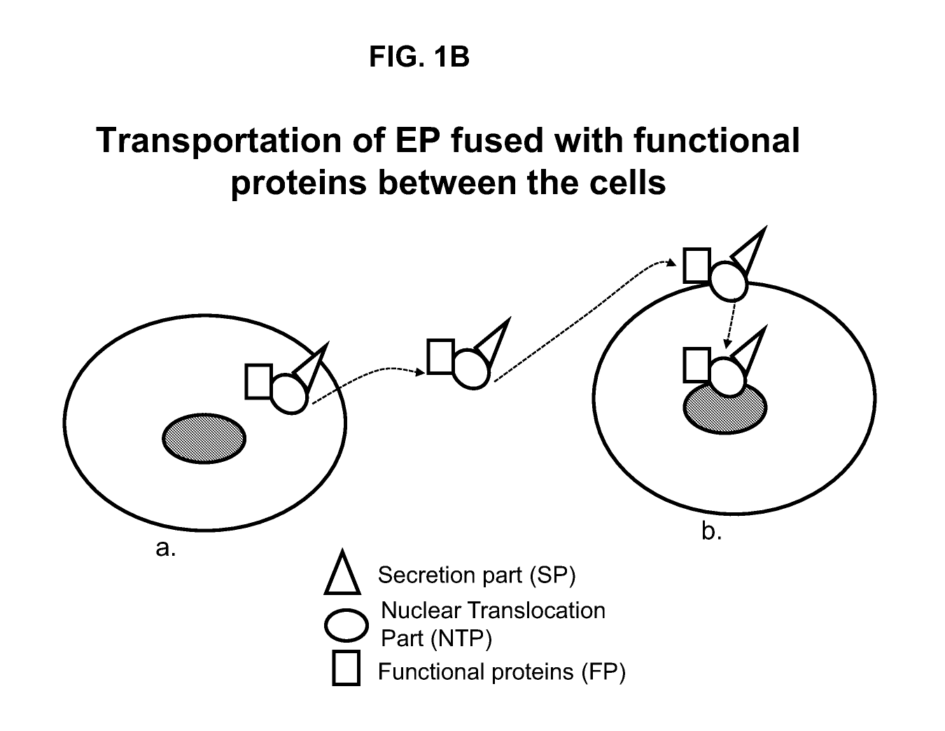

### DETAILED DESCRIPTION OF THE INVENTION

The present invention provides a protein intercellular delivery system and uses of the system in cell reprogramming, stem cell differentiation and cell-based protein therapies. The system comprises fusion proteins comprising engineered peptides for secretion from the protein expressing cells and translocation into the target cells thereof together with other functional or therapeutic proteins. The functional protein factors fused with engineered peptides are delivered for somatic cell reprogramming into pluripotency and for pluripotent stem cells differentiating into specific functional cells such as cardiomyocytes and neural cells. In particular examples the invention for cell reprogramming into pluripotency relates to fusion proteins having both pluripotent transcription factors such as Oct4, Sox2, Klf4 or c-Myc functionality, and engineered peptides as described in the claims for intercellular delivery. In particular examples of pluripotent stem cell differentiating into cardiac cells and neural cells, the invention relates to fusion protein having cardiac key transcription factors such as Tbx5, MEF2c, GATA4 or Isl1, and engineered peptide; or neural key transcription factors such as NeuroD1, Pax6 and LHX2, and engineered peptide, as described in the claims. In another particular embodiment, the invention relates to the uses of the sustained delivery of therapeutic proteins in cell-based therapies of intracellular proteins or antibodies. Other aspects of the invention will be apparent from the description and claims.

Cell penetrating peptides (CPP) have the ability to cross the plasma membrane into the cell interior in an energy and receptor-independent manner. While CPP members have high efficiency in cell translocation they have very poor capability to secrete from the expressing cells, which makes low delivery efficiency in the overall delivery process particularly requiring the processes of isolating and purifying fusion proteins from the expressing cells. Thus, this invention involves the design and development of an engineered peptide containing both secretion part and translocation part that could facilitate transporting from expressing cells into recipient cells which include a process of proteins leaving expressing cells and a process of proteins entering recipient cells. Furthermore, the present invention has its important biomedical and clinical applications including cell reprogramming and cell differentiation for regenerative medicine and cell-based protein therapies.

Recent technical breakthrough in reprogramming somatic cells into induced pluripotent stem (iPS) cells, by enforced expression of few key transcription factors, provides an autologous source of pluripotent stem cells to regenerate healthy and functional end-organ cells with great clinical prospects. Therefore, generating iPS cells using non-integrating methods is crucial to achieve the success clinical translation of this exciting technology. The current invention provides two keys to this success clinical translation in near future. One key is engineered peptide directed generation of protein-based induced pluripotent stem cells. Another key is engineered peptide directed differentiation of stem cells which significantly improves the differentiation efficiency that has been another bottle-neck toward the clinical application of induced pluripotent stem cells.

The uses of cell penetrating peptides to deliver therapeutic proteins to treat various pathological conditions, such as cancer, inflammatory diseases, oxidative stress-related disorders, diabetes and brain injury, have been widely investigated; however, the delivery efficiency and the therapeutic efficacy of proteins were not significantly improved as expected, most likely due to they are cell “penetrating” peptides, not cell “secreting” peptides. On the other hand, in the entire cell signaling network, there are more intracellular targets than extracellular targets (cell surface receptors) for targeted therapy. Today almost all targeted therapeutics were designed and developed to target extracellular sites (such as kinase inhibitors) mainly because of the difficulty of delivering therapeutic proteins (such as monoclonal antibodies) into cells.

The current invention provides a sustainable and nontoxic source of proteins of interest instead of purified proteins which otherwise often exert cellular toxicity at a high concentration against target cells. The current invention of engineered peptides comprising secretion part and translocation part has another advantage over current cell penetrating peptides that is facilitating the secretion of expressed proteins from cells. That is, our engineered peptide works as both cell “penetrating” peptide and cell “secreting” peptide. The current invention enables a high efficacy of transporting functional factors or therapeutic proteins between cells and has a great potential for clinical use.

In the present invention, using cell-based protein delivery instead of purified protein delivery has obvious advantages: one is that it is a sustained delivery and supply of functional factors needed by high efficient cell reprogramming and cell differentiation; also, there is no need for the processes of solubilization, refolding and purification of the recombinant proteins. Generally in a purified protein delivery system, the medium for cell reprogramming that contains the recombinant proteins has to be replaced several times due to the toxicity which is laborious, time consuming and most likely interfering cell reprogramming.

In the present invention, specifically engineered peptide sequences are fused with functional factors, which significantly facilitate the transportation of the reprogramming factors out of protein expressing cells and into target cells. The current system significantly increases the delivery efficiency to a level of being able to producing clinical-grade induced pluripotent stem cells in a large-scale. This system eliminates the potential risks associated with the use of viruses, DNA transfection, and potentially harmful chemicals, and thus provides a safe and efficient source of normal or patient-specific induced pluripotent stem cells for regenerative medicine. In addition, the present system provides an efficient protein delivery into a broad variety of cell types such as skin fibroblasts, cardiac fibroblasts, adult mesenchymal cells, IMR90 cells for cell reprogramming; stem cells such as embryonic stem cells and induced pluripotent stem cells for cell differentiation; and cancer cells such as DU145 (prostate), C32 (melanoma) and A541 (lung) for cell-based protein or antibody therapies.

In the present invention of the sustained protein intercellular delivery system, wherein an engineered peptide is developed comprising both cell secretion sequences and cell translocation sequences. The secretion part of the engineered peptide (Sequence ID No. 1) has 34 amino acid residues, designed based on Elliott's study (Elliott G, O'Hare P; Cell, 1997). In some embodiment, the nuclear translocation part of the engineered peptide is screened, identified and composed from the groups consisting of nuclear transduction domain and protein secretion part. Nuclear transduction domain includes, but not limited to, Drosophila homeoprotein antennapedia transcription protein (AntHD), the HIV-1 transcriptional activator TAT protein, Kaposi FGF signal sequence (kFGF), protein transduction domain-4 (PTD4), Penetratin, M918, Transportan-10, a nuclear localization sequence, a PEP-1 peptide; an amphipathic peptide; a delivery enhancing transporter, peptide sequence comprising at least 4 or more continuous arginines, and LCLRPVG, a short fragment of HBV X-protein.

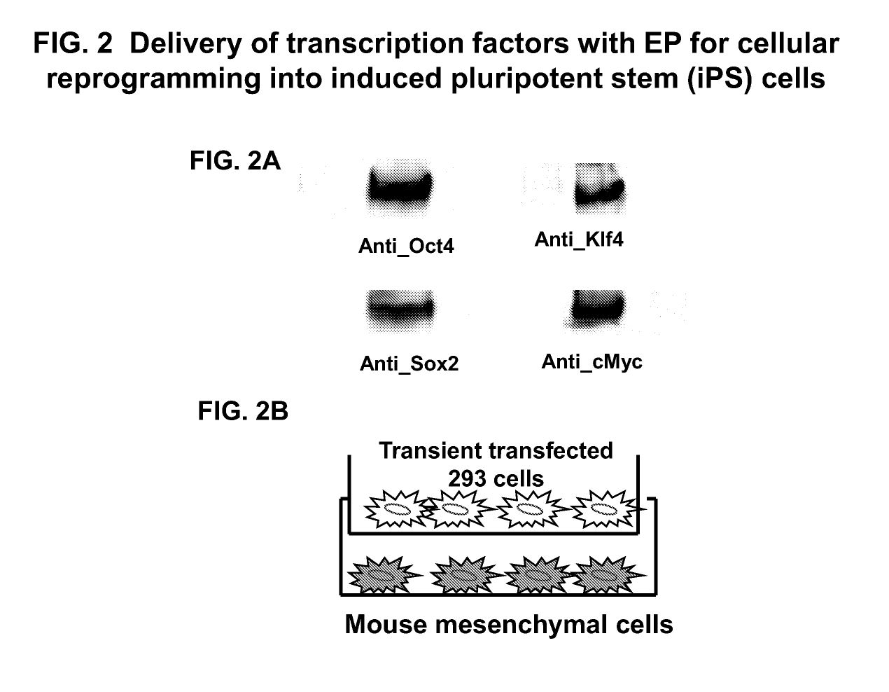

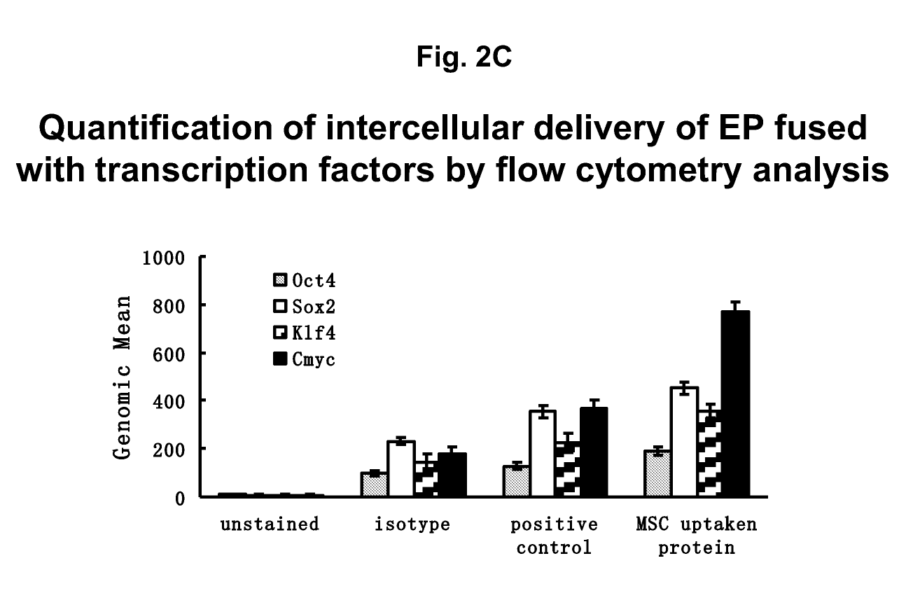

In some embodiment, sustained protein delivery system is developed, which consists of transfected cells expressing fusion proteins, a cell insert on the top where transfected cells are plated, and fusion proteins comprising protein of interest and an engineered peptide that facilitates secretion from expressing cells and translocation into somatic cells being attached on the bottom well (for example, FIG. 2B).

According to one aspect of the present invention, the transfected cell is expressing a fusion protein of a non-secreted protein such as a non-secreted transcription factor and an engineered peptide.

In some embodiment, the method comprising: contacting the non-pluripotent cell on the bottom well with one or more transfected cells expressing a transcription factor fused to an engineered peptide culturing in the cell insert; wherein the transcription factor is selected from the group consisting of a Klf polypeptide, an Oct polypeptide, a c-Myc polypeptide, and a Sox polypeptide.

In some embodiments, the engineered peptide is a peptide sequence comprising secretion part (for example, SEQ ID NO.1) and nuclear localization part (for example, SEQ ID NO. 2).

The present invention related to a safe, effective, and non-integrating strategy for reprogramming cell fate into pluripotency based on administration of sustained cell based delivery of functional proteins with an engineered peptide. Compared to other protein based reprogramming approach the present invention utilized a unique cell-based and sustainable protein delivery system to reprogram somatic cells into pluripotency so as to omit the additional step for preparation and isolation of recombinant proteins. We show that this approach can provide sustained protein delivery over several weeks and reprogram mouse, rat and human somatic cells to pluripotency with efficiencies that greatly surpass established non-integration protocols. This technology represents a safe, efficient strategy for delivering functional proteins in vivo and in vitro between cells that has broad applicability for basic research, disease modeling, drug discovery and regenerative medicine.

In order to induce the epigenetic reprogramming of the cell into a pluripotent stem cell, the reprogramming factor protein(s) may be Sox2, Klf4, Oct3/4, c-Myc, or any proteins with reprogramming activity. Accordingly, the present method of inducing pluripotent stem cell (iPS) formation avoids the use of viral or DNA-based expression vectors or the expression of reprogramming factor genes within target cells, which are known to be harmful to the host target cell and often cause cancers.

Yet another aspect of the invention relates to a method that uses at least one protein delivery vehicle expressing a transcription factor-EP (engineered peptide) fusion protein. A variety of reprogramming or differentiation factors may be introduced by the invention to generate a particular cell type with sustained protein intercellular delivery of different transcription factors with the engineered factor. Examples of cell types produced from this sustained protein delivery system of the present invention include, but are not limited to, induced pluripotent stem cells such as human induced pluripotent stem cells (hiPS), rat induced pluripotent stem cells (riPS) and mouse induced pluripotent stem cells (miPS); differentiated functional cells such as motor neurons or other differentiated neural cells, cardiomyocytes, and insulin-producing beta-cells etc.

Another aspect of the invention relates to a method of using the engineered peptides to sustainably deliver therapeutic proteins or antibodies in cell-based protein or antibody therapies. The invention opens an avenue for cell-based therapeutic proteins to be secreted from donor cells, and enter target cells and bind to the intracellular targets for therapeutic purpose. This application of the current invention is of great significance in the discovery and development of target therapeutics.

Another aspect of the invention relates to a method of using EP to sustainably deliver the transcription factors to reprogram mouse, rat and human somatic cells into induced pluripotent stem cells. And somatic cells used for reprogramming herein include, but not limited to, human mesenchymal stem cells, human IMR90, rat mesenchymal stem cells, rat skin fibroblasts, rat cardiac fibroblasts, and mouse mesenchymal stem cells.

The current invention waives the process of purifying and preparing proteins and avoids the cellular toxicity arouse by the use of a high concentration of purified proteins. The invention significantly improves the process and efficacy of delivering proteins between cells and enables the clinical-scale productions of induced pluripotent stem cells and differentiated cells. The invention also provides a great potential for improved clinical therapeutic efficacy of cell-based protein therapy.

The present invention relates to methods of using engineered peptides (EPs) to direct non-secreted protein intercellular delivery and the uses of the methods in cellular reprogramming, cellular differentiation and cell-based protein or antibody therapies. EPs contain two functional fragments: one for secretion of a protein from cells and the other for delivery of the protein into cells and thus facilitating non-secreted protein transport between cells. The current invention provides the first kind of engineered peptides which contain both a secretion fragment and a nuclear translocation fragment. Uses of EPs include cell-based sustained protein delivery of non-secreted transcription factors into somatic cells for reprogramming into pluripotent stem cells and for direct differentiation from pluripotent stem cells into other cell types. Uses of EPs also include the sustained delivery of therapeutic proteins or antibodies through in vivo transplanted cells.

### EXAMPLES

The following example, including the experiments conducted and results achieved is provided for illustrative purposes only and are not to be construed as limiting the invention.

### Example 1

Use of Engineered Peptide in Cell Reprogramming

In one example, the sustained delivery of four transcription factors Oct4, Sox2, Klf4 and C-myc, each fused with EP respectively, was implemented in a delivery system described as FIG. 2B, to induce mouse somatic cells reprogramming into induced pluripotent stem (iPS) cells. The Western blot analysis showed single bands of four fusion proteins, EP fused with four key transcription factors Oct4, Sox2, Klf4 and C-myc expressed in 293 cells. The sustained protein intercellular delivery system comprised one million of 293 cells transfected with fusion proteins of pluripotent transcription factors and EP that were plated on the cell insert, and contacting cells that were plated on the bottom well. Using flow cytometry analysis, we quantified each transcription factor (Oct4, Sox2, Klf4 or Cmyc) expressed in each cell population in the sustained protein delivery systems and found a significant high level of Oct4, Sox2, Klf4 and Cmyc fused with EP from transfected 293 cells were taken by mouse mesenchymal stem cells (mMSCs) on the bottom well.

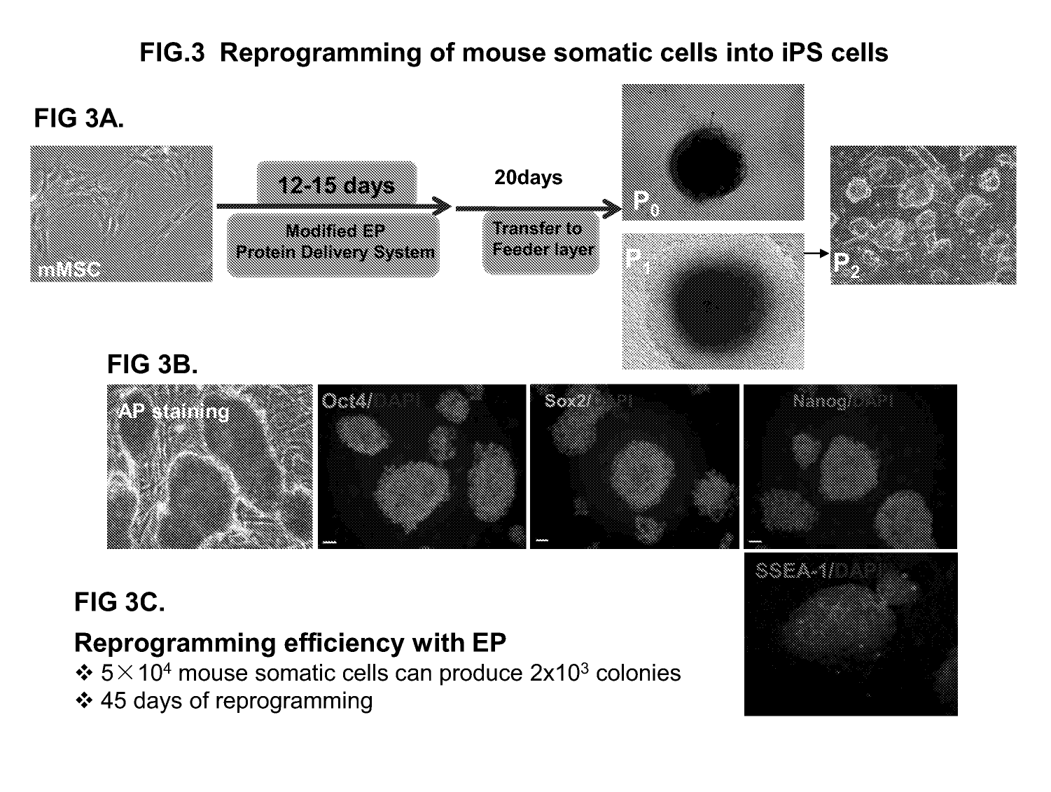

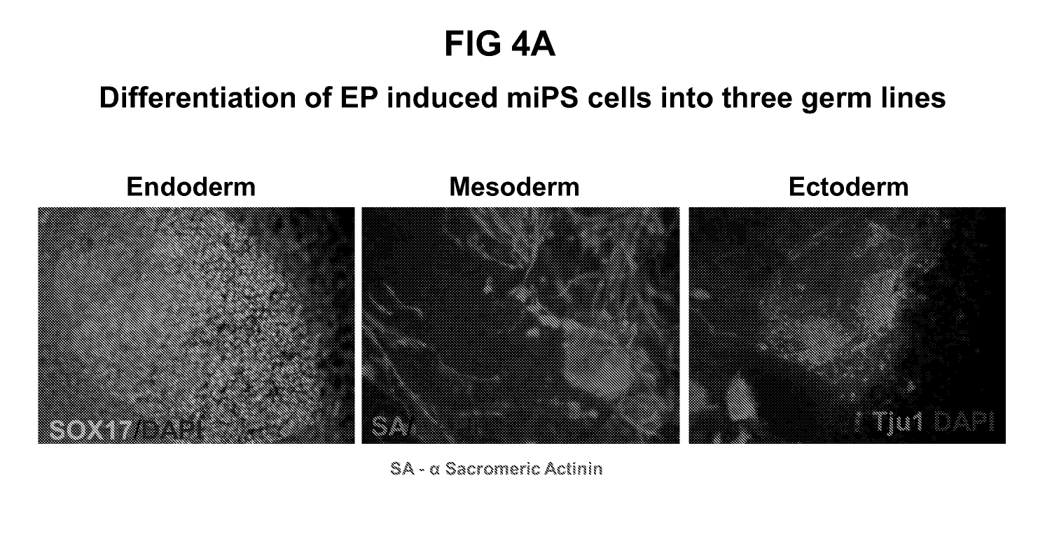

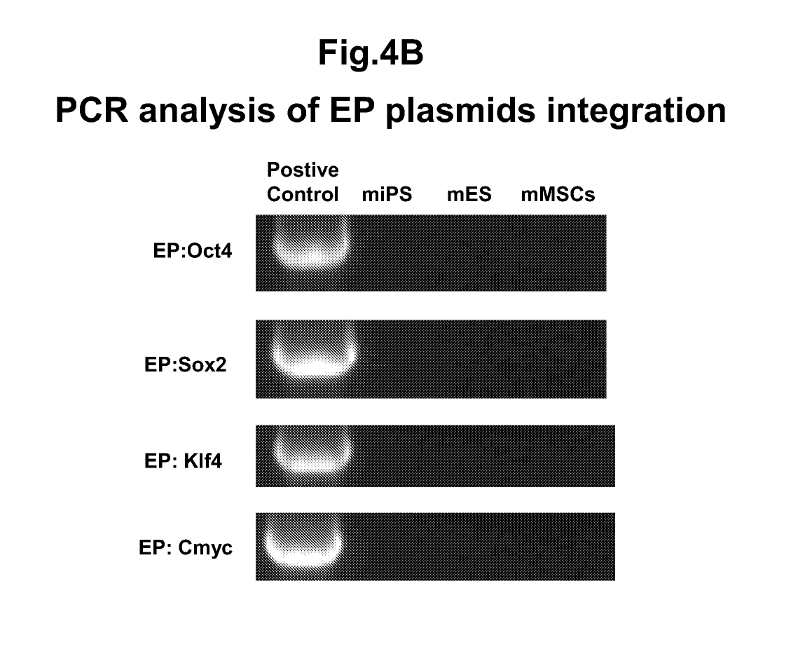

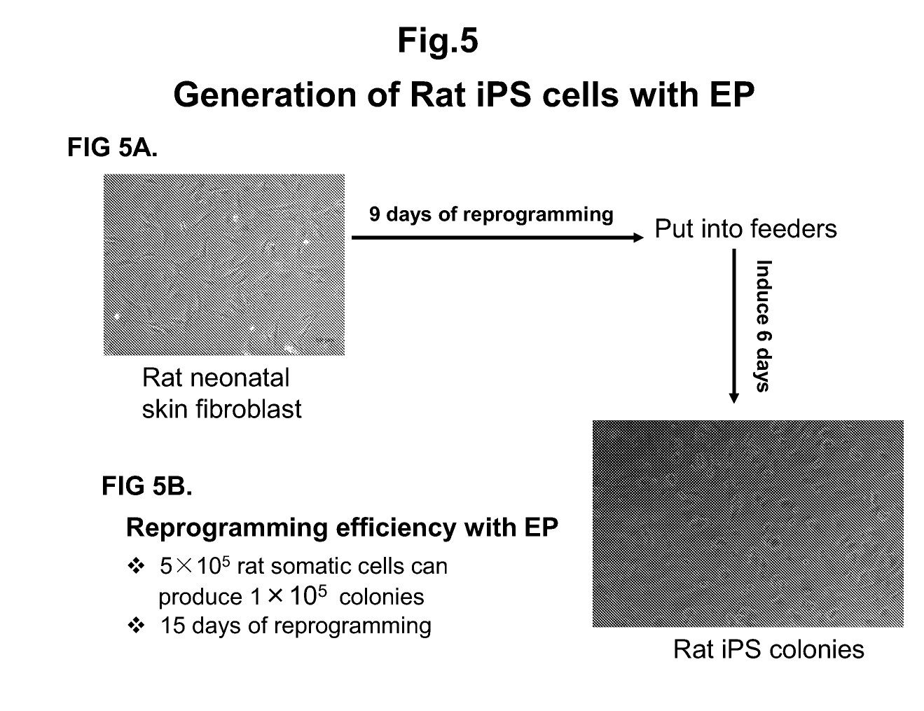

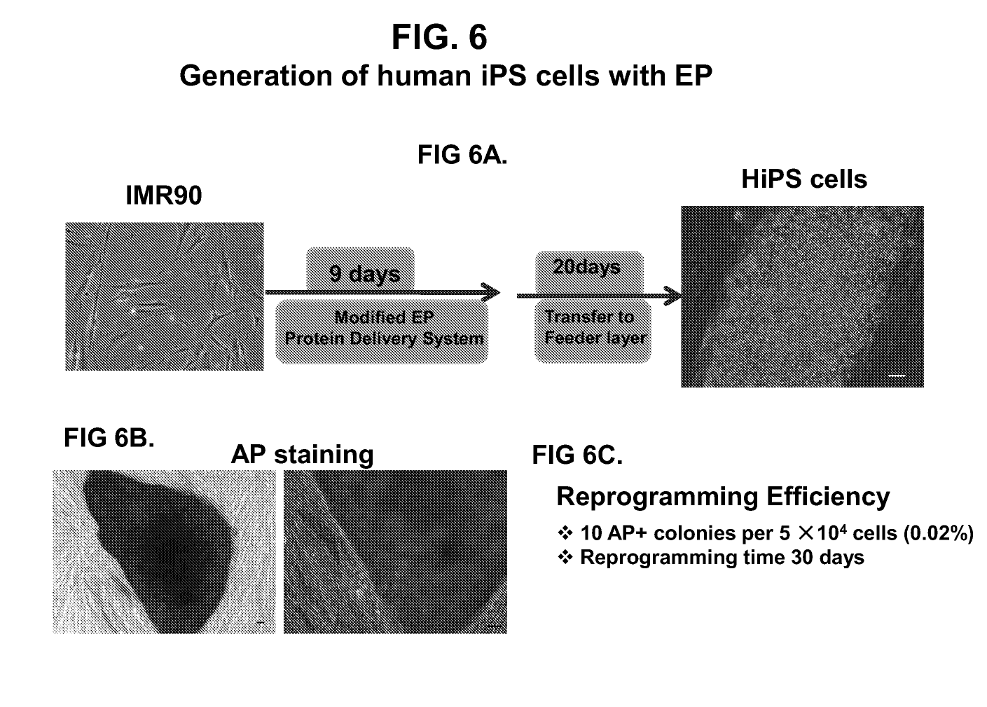

Starting on day 30, we observed mouse protein-based induced pluripotent stem (piPS) cell colonies. The representative phase contrast image of mouse mesenchymal stem cells and piPS at P0, P1 and P2 were shown in FIG. 3A. We also found piPS cells sustained a long term and homogenous self-renewal under conventional mESC growth condition. The long-term expanded piPS cells grow as compact and domed colonies that express strong AP, a typical pluripotency marker including Oct4, Sox2, Nanog and SSEA-1. DAPI staining was performed to visualize the nuclei, and the images were merged (FIG. 3B). The reprogramming efficiency of mouse piPS with the sustained protein delivery system was about 4% (FIG. 3C), significantly higher than the reprogramming rate of 0.001% from the purified protein delivery system. (Kim et al, 2009).

### Example 2

Use of Engineered Peptide in Neural Differentiation

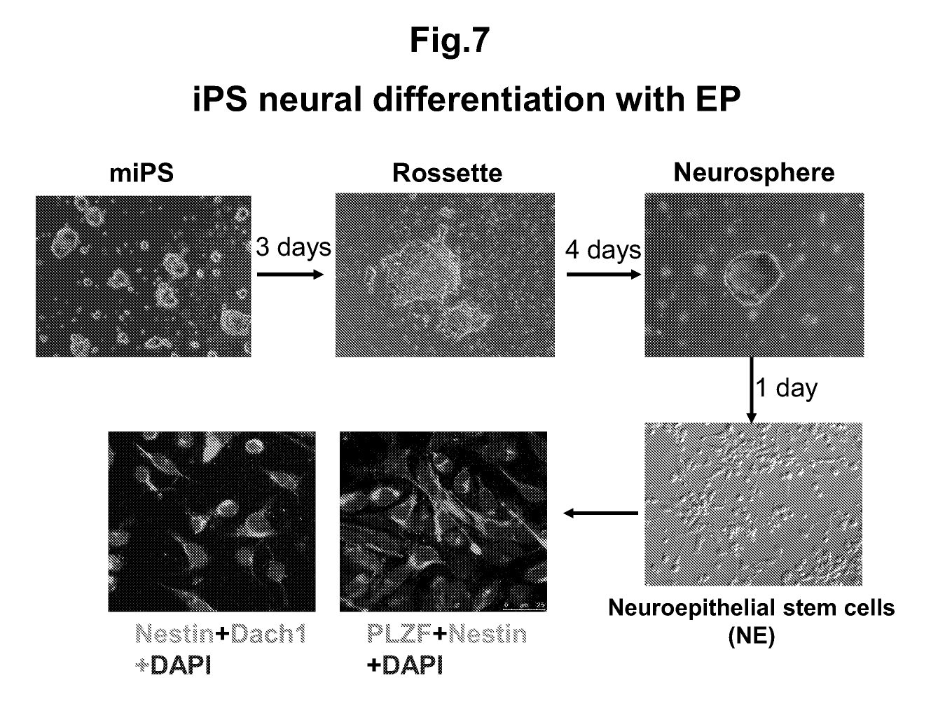

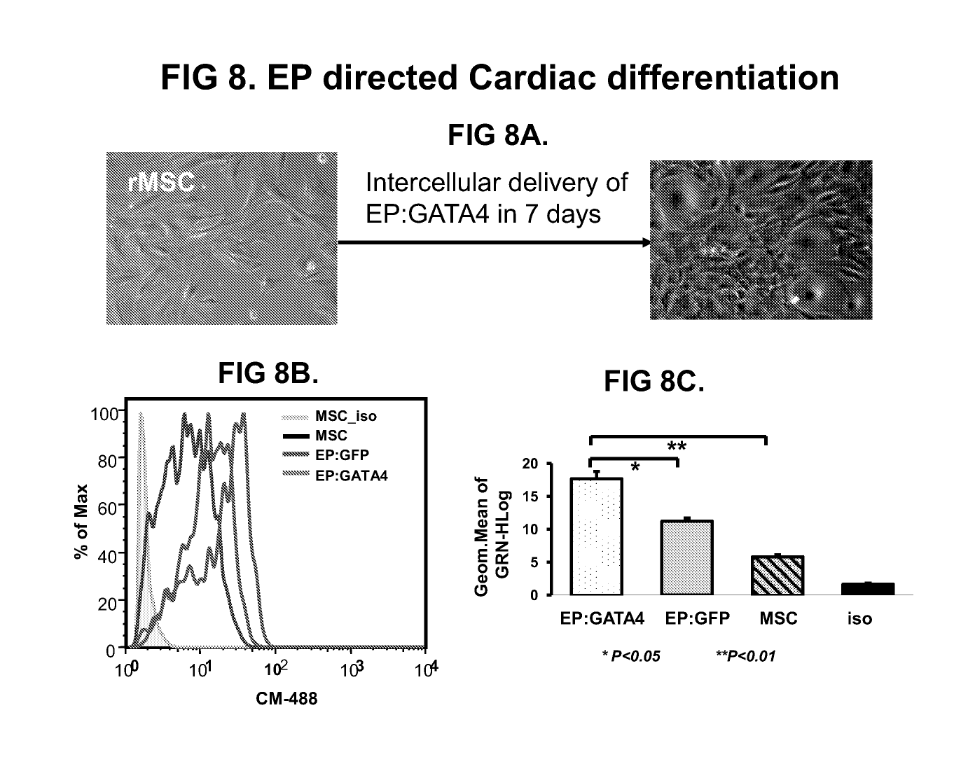

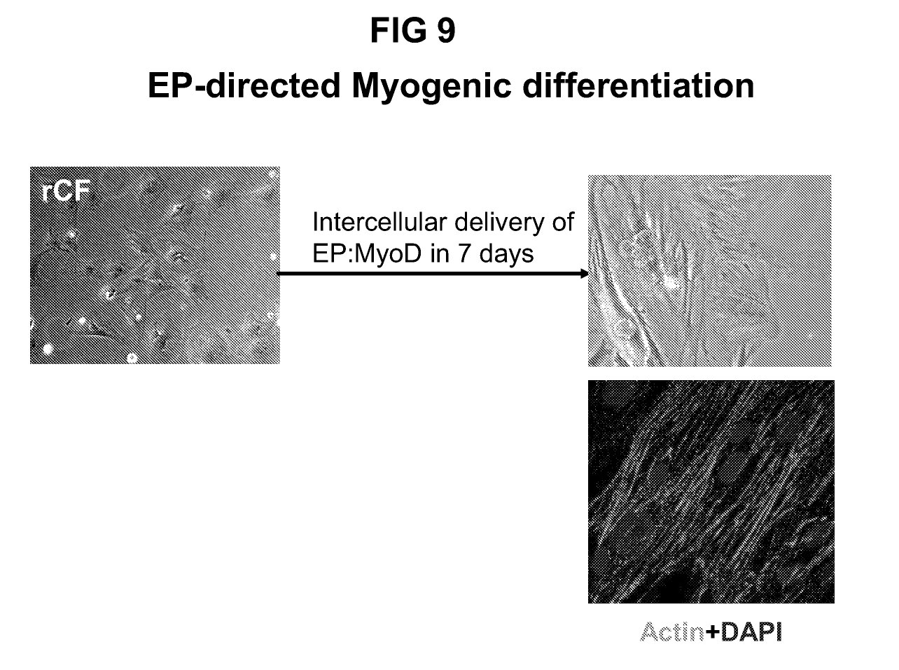

In one example of applying the invented delivery system to neural differentiation, mouse iPS cells were cultured on the bottom well in the feeder-free conditions, and one million of 293 cells transiently expressing EP fusion proteins with transcription factor Pax6 were plated on the cell insert, after 3 days of coculturing, cells plated on the cell insert were removed. Mouse iPS cells on the bottom well were observed with change of morphology in neural rossette and further differentiated into neurosphere and further differentiated into neural epithelial progenitor stem cells with NE positive markers (FIG. 7). After every stage of neural differentiation, cells were fixed with paraformaldehyde and stained with neural epithelium progenitor markers such as Nestin and Datch 1, or Nestin and PLZF (FIG. 7). All analysis suggested an efficient neural differentiation from mouse iPS cells with the EP-Pax6 fusion protein delivery system.

### Example 3

Use of Engineered Peptide in Cell-based NT3 Delivery for Treating Spinal Cord Injury

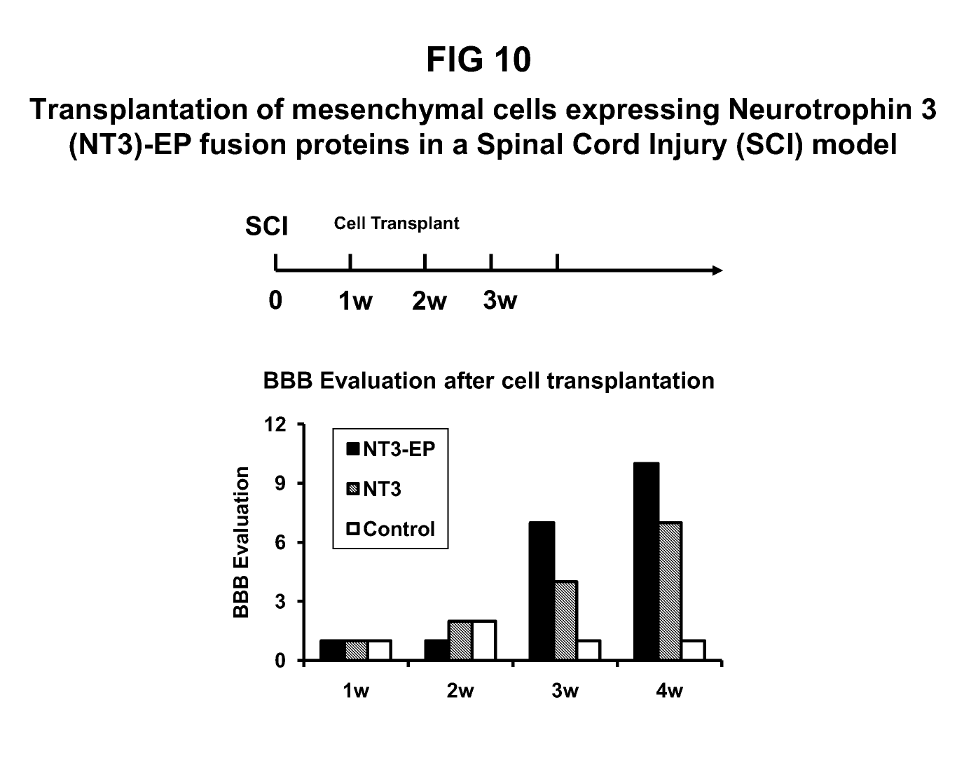

To test the in vivo delivery efficiency of the current delivery system, we employed a rat spinal cord injury. The sequence of the neural growth factor Neurotrophin 3 was fused without or with an engineered peptide sequence which has a secretion sequence (Sequence ID No. 1) and a translocation sequence comprising of 4 arginines (RRRR). The DNA constructs (NT3 and NT3-EP, respectively) were transfected with rat bone marrow mesenchymal cells. At one week after the development of spinal cord injury in SD rats (3 groups (control, NT3 and NT3-EP), 6 rats per group), the cells transiently expressing EP-NT3 fusion proteins were transplanted into the rat with spinal cord injury. Starting from the second week after cell transplantation, we found the delivery of NT3 fused with EP showed significantly improved in vivo therapeutic effect, compared to the delivery of NT3 alone and control group (FIG. 10).

Sequence No. 1:
DAATATRGRSAASRPTERPRAPARSASRPRRPVE
Sequence No. 2:
RRRRRRRRRRR

1. A sustainable intercellular protein delivery system comprising an engineered peptide linked to a functional protein.

2. The system of claim 1 comprises a method of generating engineered peptides that comprise a secretion part (SP) and a nuclear translocation part (NTP) and can be fused with functional proteins and sustainably delivery protein across the plasma membranes of cells.

3. The method of claim 2, the secretion part (SP) of an engineered peptide is a peptide sequence (SEQ ID NO.1: DAATATRGRSAASRPTERPRAPARSASRPRRPVE); and the nuclear localization part is screened, identified and composed from the group consisting of Drosophila homeoprotein antennapedia transcription protein (AntHD), herpes simplex virus structural protein VP22, the HIV-1 transcriptional activator TAT protein, Kaposi FGF signal sequence (kFGF), protein transduction domain-4 (PTD4), Penetratin, M918, Transportan-10, a nuclear localization sequence, a PEP-1 peptide; an amphipathic peptide; a delivery enhancing transporter, a short peptide (LCLRPVG) from HBV X-protein, and a peptide sequence comprising at least 4 or more continuous arginines. [(R)n, (R=Arginine, n≧4)] (for example, SEQ ID NO. 2: RRRRRRRRRRR).

4. The method of claim 2, the functional protein includes transcription factors for generating induced pluripotent stem cells (iPS), differentiation factors for differentiating pluripotent stem cells into specific cell types, and therapeutic proteins for treating diseases in patients.

5. The transcription factors of claim 4 are selected from the group consisting of a Klf polypeptide, an Oct polypeptide, a Myc polypeptide, and a Sox polypeptide.

6. The differentiation factors of claim 4 include, but not limited to, cardiac differentiation factors such as Tbx5, MEF2c, GATA4 or Isl1; neural differentiation factors such as NeuroD1, Pax6 and LHX2.

7. The therapeutic proteins of claim 4 include all proteins that can bind to any intracellular targets to inhibit, activate or regulate biological functions of a cell for therapeutic uses.

8. The method of claim 2, in the cases of generating induced pluripotent stem cells or differentiated cells, comprises a co-culture system with a cell insert (FIG. 2B), wherein transfected cells are cultured in cell inserts and the contacting mammalian cells such as fibroblasts or stem cells to be differentiated are plated in the bottom wells.

9. The induced pluripotent stem cells of claim 8 are mouse, rat or human pluripotent stem cells.

10. The contacting fibroblasts of claim 8 include, but not limited to, skin fibroblasts, cardiac fibroblasts, IMR90 and mesenchymal cells etc.

11. The differentiated cells of claim 8, wherein the contacting cells are mouse, rat or human non-pluripotent cells.

12. The system of claim 1, in the case of cell-based therapies, cells that express therapeutic proteins are transplanted in vivo.

13. The system of claim 1 is used for cell reprogramming, cell differentiation and cell-based protein therapies.

14. The system of claim 1 does not need additional step for isolation and purification of fusion proteins from transfected cells of claim 8 or from transplanted cells of claim 12.

15. The method of claim 14 includes contacting the pluripotent stem cells or non-pluripotent cells with transfected cells in the cell insert of claim 8 expressing at least one of the fusion proteins containing a transcription factor of claim 5 or a differentiated factor of claim 6, and the engineered peptide of claim 2.

16. The cell-based protein therapies of claim 13, wherein the cells are being transplanted into a living body and are capable of producing therapeutic proteins.

17. The therapeutic proteins of claims 7 and 16 comprise of enzymes, transcription factors, antibodies and other proteins of therapeutic interests.

18. The method of claim 2, the cells include transplanted cells in claim 16 and target cells such as cancer cells.
